(19) United States
(12) Patent Application Publication
(10) Pub. No.: US 2021/0174460 A1
(43) Pub. Date: Jun. 10, 2021

(54) AGRICULTURE SUPPORT SYSTEM, METHOD OF ESTIMATING PROFIT REGARDING AGRICULTURE, AND C COMPUTER-READABLE STORAGE MEDIUM

(71) Applicant: Toyota Jidosha Kabushiki Kaisha, Toyota-shi Aichi-ken (JP)

(72) Inventors: Hiroyuki Enoki, Hamamatu-shi (JP); Kazuyo Suzuki, Toyota-shi (JP); Yu Kimura, Okazaki-shi (JP)

(21) Appl. No.: 17/109,622

(22) Filed: Dec. 2, 2020

(30) Foreign Application Priority Data
Dec. 4, 2019 (JP) 2019-219849

(51) Int. Cl. G06Q 50/02; G06Q 10/08; G06Q 10/06; G06Q 30/02; G01N 33/24

(52) U.S. Cl. CPC G06Q 50/02; G06Q 10/087; G06Q 10/06375; G01N 2033/245; G06Q 30/0205; G01N 33/24; G06Q 10/06315

(57) ABSTRACT

An agriculture support system that facilitates profit estimation of even a crop that is to be newly cultivated on a land. The agriculture support system is configured as a computer system including a data input unit, a data storage unit, and an arithmetic operation unit. The arithmetic operation unit has an yield estimation part adapted to estimate a crop yield on the basis of data including characteristics and environment of a land as an estimation target so as to output yield data, a crop production data storage part adapted to store crop production data on crop production, and a profit estimation part adapted to estimate profit for a case in which the crop is produced on the basis of the yield data and crop production data so as to output profit estimation data.

### CROSS REFERENCE TO RELATED APPLICATIONS

The present application claims priority from Japanese patent application JP 2019-219849 filed on Dec. 4, 2019, the entire content of which is hereby incorporated by reference into this application.

### BACKGROUND

### Technical Field

The present disclosure relates to an agriculture support system for supporting the agriculture by providing agriculture-related information to those who are engaged in the agriculture, a method of estimating profit regarding agriculture, and a computer-readable storage medium.

### Background Art

In selecting agricultural crops to be produced, it is important to estimate profit, including sales proceeds, revenues, and profit ratios that could be obtained from production of the crops. Further, information on production planning, such as yields, farm work, work seasons, and production periods is necessary for crop production. JP 2010-257353 A discloses a program and a system for supporting farming that simulate farming by calculating sales and cost referring to past data on the expenses that have actually been required for agricultural production and by setting certain conditions.

JP H11-175609 A discloses a system for managing production and distribution of farm products that prepares production planning on the basis of such data as seeding available seasons, growth periods, harvesting seasons, and projected yields that are received from a control center. However, producing a variety of crops on different farmlands in advance to accumulate data on the crops requires enormous labor and cost as well as a long period of time.

In addition, crop yield data, which is most important data in projecting the profit in the agriculture business, is variable due to its susceptibility to environmental effects, such as weather and the nature of soil of the land, as compared to cost or the like, and thus, the yield data simulated by setting certain preconditions has not been reliable enough to be available for practical use.

Meanwhile, promotion of utilization of abandoned cultivated land has been sought in the agriculture sector. For example, JP 2014-098993 A discloses a device, method, and program for cultivated farm field determination, in which farmland where no planting is assumed to be performed is identified on the basis of satellite images. However, JP 2014-098993 A does not disclose, either, production planning, including crop selection, yields, farm work, work seasons, and production periods, which are important factors in promoting the utilization of abandoned cultivated land, or estimation of profit, including sales, costs, and revenues.

As described above, it has been difficult to accurately evaluate the information on what kind of crop should be selected for cultivation on the existing farmland, newly cultivated land, or abandoned cultivated land. For example, although farm product producers, newly engaged farmers, production contractors, or production instructors may be able to substantially estimate the yields of and profits from the crops that have been produced on the land, it is difficult for them to estimate yields of and profits from the crops that have never been produced on the same land or to compare and examine a plurality of new crops. It is also difficult for land owners, land managers, or land lessees (including municipalities, agricultural material companies, and nursery companies) to accurately evaluate farmland.

### SUMMARY

The present disclosure provides an agriculture support system that facilitates profit estimation of even a crop that is to be newly produced on a land, a method of estimating profit regarding agriculture, and a computer-readable storage medium.

In view of the foregoing, an agriculture support system according to the present disclosure is configured as a computer system including a data input unit, a data storage unit, and an arithmetic operation unit. The arithmetic operation unit has a yield estimation part adapted to estimate a yield of a crop on the basis of data including characteristics of a land as an estimation target and environment of the land so as to output yield data, a crop production data storage part adapted to store crop production data on the crop production, and a profit estimation part adapted to estimate profit for a case in which the crop is produced on the basis of the yield data and the crop production data so as to output profit estimation data.

The present disclosure provides an agriculture support system that facilitates profit estimation of even a crop that is to be newly produced on a land.

### BRIEF DESCRIPTION OF THE DRAWINGS

FIG. 1 is a block diagram illustrating the overall configuration of an agriculture support system 1 according to a first embodiment;

FIG. 2 is a flowchart explaining the operation of the agriculture support system 1 according to the first embodiment;

FIG. 3 is a block diagram illustrating the overall configuration of the agriculture support system 1 according to a second embodiment;

FIG. 4 is a flowchart explaining the operation of the agriculture support system 1 according to the second embodiment;

FIG. 5 is a block diagram illustrating the overall configuration of the agriculture support system 1 according to a third embodiment; and

FIG. 6 is a flowchart explaining the operation of the agriculture support system 1 according to the third embodiment.

### DETAILED DESCRIPTION

The present embodiment will be described below with reference to the attached drawings. In the attached drawings, some elements having the same functions may be denoted by the same reference numerals. It should be noted that although the attached drawings illustrate the embodiments and examples based on the principle of the present disclosure, the embodiments and examples are presented to support the understanding of the present disclosure, and should not be construed as limiting the present disclosure. The present specification merely provides typical examples, which do not limit the scope of the claims or applicable examples of the present disclosure in any meaning.

The embodiments of the present disclosure are sufficiently detailed to the extent that those skilled in the art can carry them out, but it must be noted that other implementations and aspects are also available, and the configuration and structure of the embodiments may be modified and the elements may be replaced with others in various manners without departing from the scope and spirit of the technical idea of the present disclosure. Thus, the following descriptions should not be construed as limiting the present disclosure.

### First Embodiment

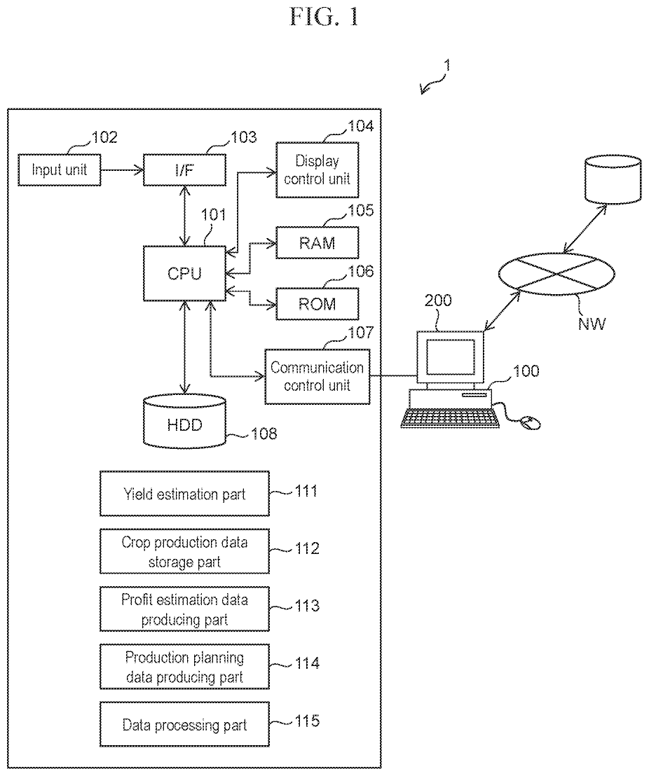

Next, an agriculture support system according to a first embodiment will be described with reference to FIG. 1, etc.

FIG. 1 is a block diagram illustrating the overall configuration of an agriculture support system 1 according to the first embodiment. The agriculture support system 1 includes a computer 100 capable of accessing agriculture-related big data via a network NW, and a display 200. The computer 100 is capable of producing, on the basis of the agriculture-related big data, data on what kinds of crops generate what levels of profits in areas as an estimation target and on production planning for the crops.

As an example, the computer 100 includes a CPU 101, an input unit 102, an interface (I/F) 103, a display control unit 104, a RAM 105, a ROM 106, a communication control unit 107, and a hard disk drive (HDD) 108. The CPU 101 is an arithmetic control circuit that addresses various pieces of arithmetic processing, control, and order in the computer 100. The input unit 102 is a device for receiving instructions and selections from users, such as a keyboard, mouse, and touch panel. The display control unit 104 controls the display of estimation results obtained through analysis and arithmetic operation of various data obtained via the network NW.

The HDD 108 stores a computer program for executing profit estimation processing and production planning data production. This computer program defines processing procedures to virtually implement, in the computer 100, a yield estimation part 111, a crop production data storage part 112, a profit estimation data producing part 113, a production planning data producing part 114, and a data processing part 115. The operations of the parts 111 to 115 will be described later.

The yield estimation part 111 estimates what level of yield would be obtained from a crop produced on a land as an estimation target (such as existing farmland, newly developed land, abandoned cultivated land, and unused land) on the basis of various data. The yield estimation part 111 may estimate, by data mining, the yields of one or more crops produced on one or more lands by selecting representative data for an estimation model from one or more environmental data, such as average values of the weather and the nature of soil, and yield data, such as average values of the crop yields. Herein, examples of the environmental data include soil data and environmental data. The soil data may be obtained from the GIS data (http://nrb-www.mlit.go.jp/kokjo/inspect/landclassification/dowvnload/) of the National Land Information Division or the like, and the environmental data from the WAGRI (https:/api.wagri.net/), the Automated Meteorological Data Acquisition System (wwv.jma.go.jp) of the Japan Meteorological Agency, or the like. Examples of the estimation results obtained by the yield estimation part include those in the crop situation research (http://www.maff.go.jp/j/tokei/kouhyou/sakumotu/index.html) of the Ministry of Agriculture, Forestry and Fisheries. Data mining is a method for big data analysis using machine learning or the like, whose specific examples include Random Forest, GLMNET Lasso, and PLS. Examples of data to be obtained include yield estimates of each crop and land per unit area, the ranking of crops and lands, categories based on the yield estimates and the ranking, and heat maps.

The crop production data storage part 112 is a storage that stores crop production data. Examples of the crop production data include data on sales, costs, farm work seasons, production periods, and harvesting seasons regarding the crops. In addition, the crop production data storage part 112 stores data on crops by different lands. The crop production data storage part 112 may store all the available crop production data in the hard disk drive 108 in advance or data appropriately forwarded from an external database (server) (not shown). Data on crop sales may be obtained from, for example, the regional wholesale market prices in the Portal Site of Official Statistics of Japan, “e-Stat.” Data on cost of crop production may also be directly or indirectly obtained from the e-Stat. Further, data on farm work seasons and production periods may be obtained from the planting information.

Data on cost of crop production may include farming expenses, farming income, farming balance, working hours, and the like. Further, the data on cost of crop production may be obtained from expenses of crop production, farming gross profit, farming expenses, farming income, farming balance, working hours, and the like. More specifically, the data on cost of crop production may be calculated from agricultural income, agricultural miscellaneous income, expenses for shipping to and receiving from markets, labor cost, cost of seeds and seedlings, fertilizer cost, agricultural chemical cost, material cost, utility cost, agricultural implement cost, cost of buildings for agriculture use, horticultural facility cost, rent, operation consignment fee, land improvement cost, water charge, packaging cost, packing cost, shipping cost, etc.

The profit estimation data producing part 113 estimates, on the basis of the aforementioned yield estimation data and crop production data on the selected crop, the profit to be generated when the crop is cultivated on the land or the profit-related items, and outputs them as profit estimation data. For example, the profit estimation data producing part 113 uses, as input data, the yield estimates of the crops per unit area, sales estimates per unit yield, and cost estimates per unit area for each combination of crops and lands to estimate the sales per unit area, profit, and other profit-related data.

As an example, the sales of each crop per unit yield on the supermarkets and wholesale markets are applied to the yield estimates of each crop and each land per unit area, so as to estimate profit generated from the land and the crop. The wholesale markets may also include those in the neighborhood as well as major wholesale markets across the country.

Here is an example of the Nagoya Central Wholesale Market. The monthly average or median values of sales of crops per unit yield and sales proceeds of crops per unit yield in harvesting seasons may be obtained as sales data. When it is assumed that the average sales of tomatoes per unit yield on the Nagoya Central Wholesale Market are 357 yen/kg, and the yield estimate of tomatoes per 10a (per unit area) is 6,167.3 kg/10a, the estimated sales (per unit area) are 2.202 million yen/10a.

Further, as an example, the profit may be estimated from the aforementioned sales and the costs of crops per unit area that are obtained from the e-Stat, municipalities, research institutes, and the like. For example, when the national average cost of tomatoes per 10a is assumed to be 1.369 million yen/10a using the information on the national average cost or the costs by prefecture in the e-Stat, the profit becomes 0.833 million yen/10a on the basis of the above estimated sales of 2.202 million yen/10a.

The production planning data producing part 114 has a function of producing production planning data on the basis of the aforementioned yield estimation data and crop production data. The production planning data includes data on the yield estimate of each crop produced on each farmland per unit area, the farm work season, production period, working hours, and agricultural off-season. In particular, for production planning for a plurality of crops over a plurality of years, as in rotational cultivation or double cropping, the crop production data on the crops is prepared for the plurality of years as a basis. When the agricultural off-season is provided, data on the agricultural off-season is also included in the production planning data. In production planning for a plurality of crops over a plurality of years, the production planning data may include yield estimates per unit area, farm work, work seasons, production periods, working hours, and agricultural off-seasons regarding each combination of farmlands and crops for each year.

The production planning data may include, for example, data on the farm work, operation season, production period, working hours, and agricultural off-season regarding each crop based on the planting information. The following are examples of production planning for carrot, Chinese cabbage, and eggplant:

Crop: carrot
Cultivation method: spring seeding
Production period: early January-early June
Working hours: 118.17 hours
Agricultural off-season: mid-June-late August
Seeding: early January
Tunnel installation: early January
Harvesting season: mid-May
Crop: Chinese cabbage
Cultivation method: summer seeding and transplanting
Production period: early September-mid-November
Working hours: 93.66 hours
Agricultural off-season: late November-early March
Seeding: early September
Harvesting season: mid-December
Crop: eggplant
Cultivation method: premature cultivation under tunnel
Production period: mid-March-early September
Working hours: 1049.06 hours
Seeding: early January
Heating: early January
Raising seedling: early January
Grafting: mid-February
Settled planting: mid-March
Tunnel installation: late March
Harvesting season: early May

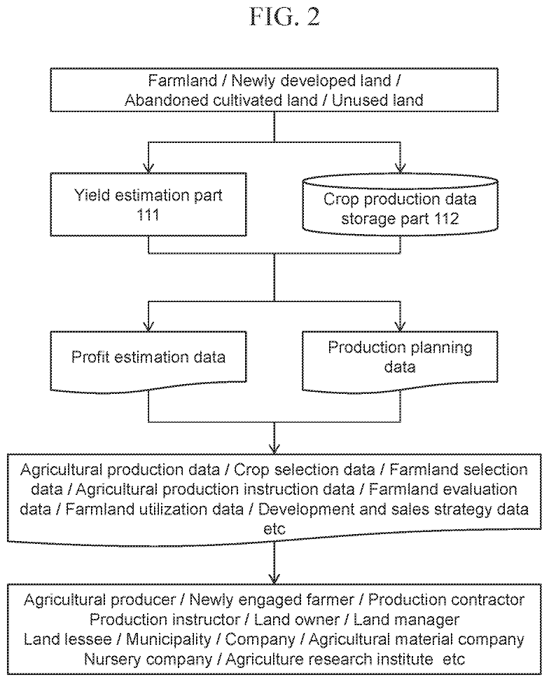

The data processing part 115 has a function of processing the profit estimation data and production planning data that are produced to further produce various data. Examples of the various processed data include agricultural production data, crop selection data, farmland selection data, agricultural production instruction data, farmland evaluation data, farmland utilization data, and development and sales strategy data With such data provided, agricultural producers, newly engaged farmers, production contractors, production instructors, and the like are able to easily compare and examine the production of a plurality of crops on a plurality of farmlands, thereby enabling the selection of more profitable crops and farmlands, appropriate instructions on production, promotion of smooth purchase, sales, and leases of farmlands based on the farmland evaluation considering the profitability, and development and sales promotion of agricultural materials and seeds and seedlings considering the relations between the crops and the farmlands (see FIG. 2).

The crop selection data is data for use in selecting crops for one or more targeted farmlands and is provided to agricultural producers, newly engaged farmers, production contractors, production instructors, and the like. The crop selection data may include agricultural production information containing profit estimates and production plans for one or more crops to be produced on one or more farmlands. Specifically, the crop selection data may include sales estimates, profit estimates, and costs of one or more crops to be produced on the targeted farmlands per unit area, the ranking of one or more crops, categories based on the profit estimates and the ranking, and the yield estimates per unit area, farm work, operation seasons, production periods, working hours, and agricultural off-seasons regarding one or more crops. When the production planning for a plurality of crops over a plurality of years is included, the crop selection data may include yield estimates per unit area, farm work, operation seasons, production periods, working hours, and agricultural off-seasons regarding one or more crops for each year.

The farmland selection data is data provided to agricultural producers, newly engaged farmers, production contractors, and production instructors to be used for selecting farmlands for one or more targeted crops, the data including profit estimates and production planning for one or more crops to be produced on one or more farmlands. Specifically, the farmland selection data includes sales estimates, profit estimates, and costs of the targeted crops to be produced on one or more farmlands per unit area, the ranking of the crops to be produced on one or more farmlands, categories based on the profit estimates and the ranking, and the yield estimates per unit area, farm work, operation seasons, production periods, working hours, and agricultural off-seasons regarding the crops to be produced on one or more farmlands. For production planning for a plurality of crops over a plurality of years, the farmland selection data may include, in addition to the above items, sales estimates, profit estimates, and costs of the crops to be produced on one or more farmlands per unit area based on the production planning data and profit data, the ranking of the crops to be produced on one or more farmlands, categories based on the profit estimates and the ranking, and the yield estimates per unit area, farm work, operation seasons, production periods, working hours, and agricultural off-seasons regarding the crops to be produced on one or more farmlands for each year.

The agricultural production instruction data is data for instructions on production to be provided to production contractors (such as companies, research institutes, and retailers) and production instructors (such as companies, farmers' cooperatives, municipalities, and research institutes). Specifically, when a crop and a farmland as a target are specified, a production instruction manual prepared after selecting the crop and farmland is provided as the agricultural production instruction data in addition to the agricultural production data, crop selection data, and/or farmland selection data. Further, when a crop as a target is specified, the production instruction manual prepared after selecting the crop and farmland is provided in addition to the farmland selection data.

The farmland evaluation data is data for presenting the results of evaluation of farmlands conducted, for example, from a profitability point of view, to land owners, land managers, and land lessees. When a farmland and/or a crop as a target is specified, the farmland is evaluated from a profitability point of view calculated after selecting the crop and farmland, and the evaluation results of the farmland are presented as the farmland evaluation data, in addition to the agricultural production data. The land owners herein may include lease holders, farmers, non-farmers who own lands, municipalities, companies, retailers, and farmers' cooperatives. The land managers may include real estate agents, lease holders, farmers, non-farmers who own lands, municipalities, companies, retailers, and farmers' cooperatives. The environmental data includes soil data, environmental data, and the like. Examples of the soil data include the GIS data (http://nrb-www.mlit.go.jp/kokjo/inspect/landclassification/download/) of the National Land Information Division, and those of the environmental data include the WAGRI (https://api.wagri.net/) and the Automated Meteorological Data Acquisition System (www.jma.go.jp) of the Japan Meteorological Agency. Examples of the yield data include the crop situation research (http://www.maff.go.jp/j/tokei/kouhyou/sakumotu/index.html) of the Ministry of Agriculture, Forestry and Fisheries. Data mining is a method for big data analysis using machine learning or the like, whose examples include Random Forest, GLMNET Lasso, and PLS. Examples of data to be obtained include yield estimates of each crop and land per unit area, the ranking of crops and lands, categories based on the yield estimates and the ranking, and heat maps. Farmers, companies, agricultural schools, research institutes, farmers' cooperatives, companies, retailers, and the like may be included.

The farmland utilization data is data on utilization of farmlands that is provided to individuals, corporations, organizations, and the like who want to utilize farmlands, such as municipalities and companies. Specifically, when farmlands and/or crops as a target are specified, the farmland utilization data prepared after selecting the crops and farmlands is provided in addition to the agricultural production data, crop selection data, and/or farmland selection data.

The development and sales strategy data is data on research and development and sales strategy of agricultural materials and seeds and seedlings that are required for cultivation of crops, the data being provided to agricultural material companies, nursery companies, agriculture research institutes, and the like. When farmlands and/or crops as a target are specified, the development and sales strategy data on the agricultural materials and seeds and seedlings prepared after selecting the crops and farmlands is provided, in addition to the agricultural production data, crop selection data, farmland selection data, and the like. The agricultural material companies herein may include agricultural chemical companies, fertilizer companies, agricultural machinery companies, and the like as well as agricultural material companies that provide agricultural implements. The nursery companies may include production and sales companies of seeds and seedlings as well as seeds and seedlings developing companies. The agriculture research institutes may include the National Agriculture and Food Research Organization. Agricultural Experiment Stations. and the like.

The agricultural producers herein include farmers, agricultural schools, farming business operators, farmers having a second job, agricultural business entities, agricultural service operators, agricultural corporations, farmers' cooperatives, and the like. Further, the newly engaged farmers include successors of farmers, entrepreneurs, new graduates engaged in farming, those having their main job shifted to farming, and the like. The production contractors include companies, research institutes, retailers, and the like. The production instructors include companies, farmers' cooperatives, municipalities, research institutes, and the like.

The crop herein refers to grain, vegetables, fruit, flowers, etc. The crop is not limited to one crop, but may also include a plurality of crops. The farmland includes land under consideration for future use for agricultural production, such as newly developed land, abandoned cultivated land, and unused land as well as existing farmland already in use for agricultural production. The farmland is not limited to one farmland, but may also include a plurality of farmlands.

To improve profitability in agriculture, it is important to estimate data on sales, profit, and the like to be obtained through production of a plurality of crops on a plurality of farmlands (hereinafter collectively referred to as the “profit estimation data”), so as to compare and examine the profit generated from the plurality of crops. However, the yield data, which is most important data in calculating the profit, significantly varies because it is highly susceptible to the production environment, as compared to such information as the cost required for the crop production and the market prices of the crops per yield unit, and therefore, when new crops are produced, it is necessary to experimentally produce candidate crops to evaluate their adaptability to the environment. Meanwhile, there are a wide variety of crops ranging from grain (rice plant, wheat, barley, etc.), vegetables (soybean, peanut, peas, tomato, eggplant, bell pepper, paprika, potato, sweet potato, cabbage, lettuce, Chinese cabbage, radish, broccoli, green onion, onion, cucumber, pumpkin, spinach, carrot, burdock, etc.), fruit (apple, peach, pear, orange, grapes, strawberry, melon, watermelon, etc.) to flowers (chrysanthemum, calla lily), etc., and therefore, experimentally producing a variety of these crops for comparison and examination requires huge labor and cost and many years, which has been a huge obstacle in improving the profitability in the agriculture.

In addition, the increasing abandoned cultivated land has recently been a problem. The factors of such increasing abandoned cultivated land include lack of crops that could boost profit, few new farmers, and the like.

To address such a problem of abandoned cultivated land, various measures have been taken. For example, in Hokkaido, which has been experiencing deteriorating farming business environment due to falling milk prices and surging feed grain prices and aging of farmers, abandoned cultivated land has been increasing. However, with such incentives as subsidies for promoting utilization of dilapidated farmland and the like, the production has been shifted from the existing crops to highly profitable Fagopyrum tataricum, thereby encouraging revitalization of abandoned cultivated land.

Similarly. Ishikawa prefecture has also been facing the problem of the increasing abandoned cultivated land, since farmers have left the agriculture sector due to declining demand for leaf tobacco. However, the production has been shifted from the existing crops to more profitable potato, black cabbage, red radish, etc., to increase profit, so that revitalization of abandoned cultivated land has been promoted.

As can be seen in these examples, the revitalization of the agriculture sector has been advanced by shifting the production from less profitable existing crops to highly profitable other corps. On the other hand, selecting highly profitable crops is not easy, and in particular, it is difficult to compare and examine a plurality of crops and farmlands. This has been a major challenge in promoting the revitalization of abandoned cultivated land and expansion of newly developed land.

Further, although those who newly start farming need to select farmlands and crops, those who are engaged in the agriculture have limited information on farmlands, and thus, find it difficult to compare and examine a plurality of crops and farmlands. In particular, when crops that have never or hardly been produced in the region are to be produced, it is difficult to obtain the information on the crops, such as farm work seasons, production periods, and harvesting seasons as well as profitability. Particularly, for production of a plurality of crops over several years in rotational cultivation or the like, the production periods, yields, and profits of the crops need to be examined in combination. However, it is difficult to obtain such information, which has been among factors hampering the growth in the number of new farmers.

Furthermore, to improve the agricultural productivity, appropriate farm work, particularly, introduction of latest technology is important, and instructions on production play a key role. However, production instructors may typically have information on crops that they have handled, while they may lack in the information on new crops. It is particularly difficult to compare a plurality of crops and farmlands to provide appropriate instructions.

Moreover, to increase the agricultural productivity through establishing a new production region or the like, development and sales of agricultural materials or seeds and seedlings conducted by agricultural material companies or nursery companies are essential. Meanwhile, in some cases of production of crops that have never been produced, information by climate zone may be used, but the information is limited, and such lack of information has been a huge obstacle in the development and sales of agricultural materials or seeds and seedlings. In addition, the promotion of farmland utilization is also important for improving the agricultural productivity, but a farmland evaluation scheme based on productivity or profitability is not in place. Thus, the evaluation relies on intuition and experience of land owners and land managers with limited information, which is a cause of insufficient progress in farmland utilization at present.

To deal with the situation, in the agriculture support system of the first embodiment, as described above, the CPU 101 included in the arithmetic operation unit executes the aforementioned program to estimate the crop yield on the basis of data including the characteristics and the environment of the land as an estimation target, and further, to estimate the profit generated when the crop is produced on the basis of the yield data and the crop production data. In addition, crop production planning is also conducted on the basis of the yield estimates and the crop production data. The profit estimation data and the crop production planning data produced as such are presented to those who are engaged in the agriculture. Further, the profit estimation data and production planning data may be processed into different data to be output. In this manner, according to the present embodiment, cultivation on new lands or of new crops can be performed on the basis of the production planning based on data, while profit estimation is conducted.

Further, the system of the present embodiment is capable of providing effective information not only to farmers who have already been actually cultivating crops, but also to newly engaged farmers or land owners and companies who are planning on effective use of their own lands either by using them on their own or allowing other entities to use them. The system of the present embodiment can accumulate such information as profitability evaluation of multiple farmlands to construct the accumulated information as a database. The database is publicized through the Internet or the like to be made available for search and browse, so that those who are considering starting agriculture business by leasing lands, for example, can browse the database and consider leasing farmlands. Those who are considering leasing lands can search information on the profitability of lands and crops, regions, and land areas, when browsing the database. In this manner, with the use of the evaluation information providing system, in which the information on the profitability evaluation is constructed as a database, land owners and land managers, and land lessees can be matched.

### Second Embodiment

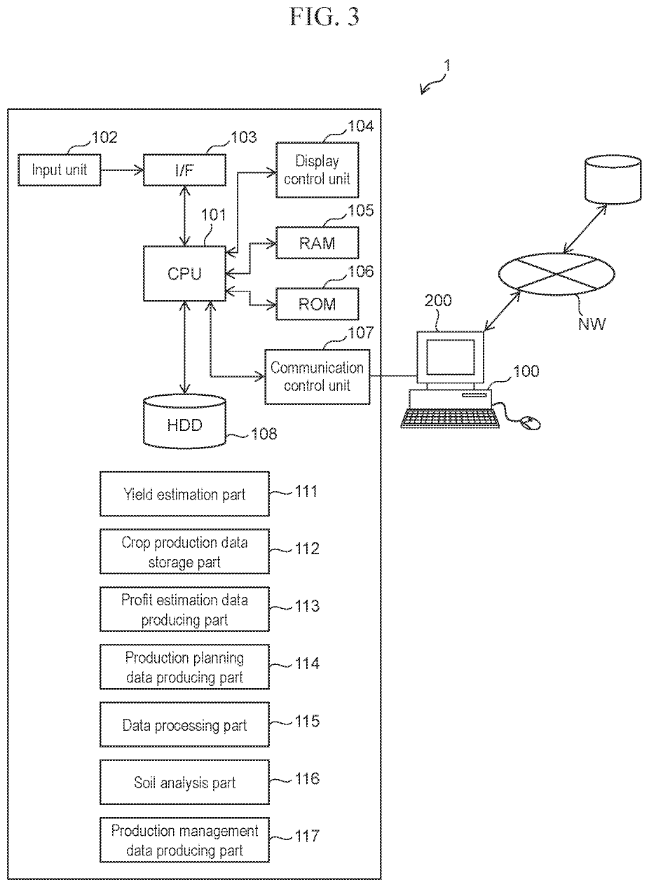

Next, the agriculture support system according to a second embodiment will be described with reference to FIG. 3, etc. FIG. 3 is a block diagram illustrating the overall configuration of the agriculture support system 1 according to the second embodiment. The system 1 of the second embodiment differs from that of the first embodiment in the computer program stored in the HDD 108 and the portion that is virtually implemented by the computer program. The constituent elements in FIG. 3 that are the same as those in FIG. 1 are denoted by the same reference numerals, and the overlapping descriptions will be omitted below.

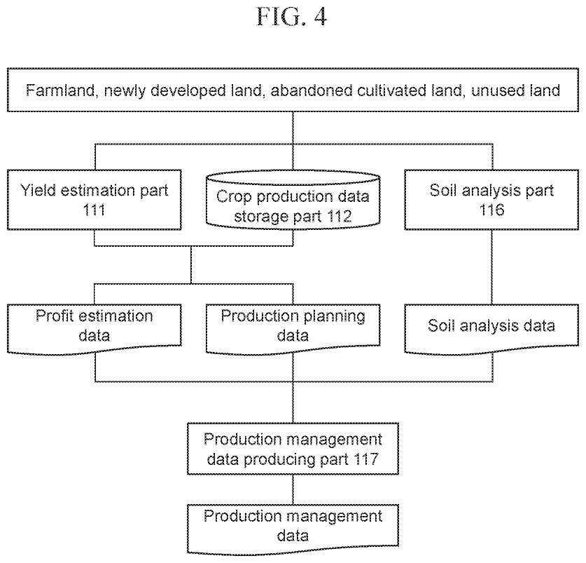

The computer program of the system 1 of the second embodiment includes a soil analysis part 116 and a production management data producing part 117 in addition to the same functions as those of the first embodiment. The soil analysis part 116 analyzes the characteristics of the soil of a targeted land, and outputs the analysis results as soil analysis data. Meanwhile, the production management data producing part 117 has a function of producing production management data on production management for producing a crop on a land in accordance with the selected crop and land, and the profit estimation data, production planning data, and soil analysis data on the crop to be produced on the land (see FIG. 4).

The soil analysis part 116 may also be implemented by using known systems, such as the “SOFIX” (URL:https://www.kuritabunseki.co.jp/?page_id=7414) of Kurita Analysis Service Co., Ltd., the “Soil Analysis” (URL:http://mirai-zou.co.jp/) of Miraizou Co., Ltd., and the “Simple Soil Diagnosis” (http://www.maff.go.jp/j/seisan/kankyo/hozen_type/h_sehi_kizyun/tottori01.html) of the Ministry of Agriculture, Forestry and Fisheries. Further, the production management data producing part 117 may also be implemented by using known systems, such as the “Housaku Keikaku” (an agricultural IT management tool) (https://www.toyota.co.jp/housaku/) of Toyota Motor Corporation, the “Akisai” (FUJITSU Intelligent Society Solution) (https://jp.fujitsu.com/solutions/cloud/agri/) of Fujitsu Limited, the “Cultivation Navi” (https://agri.panasonic.com/saibai/) of Panasonic Corporation, and the “KSAS” (Kubota Smart Agri System) (https://ksas.kubota.co.jp/) of Kubota Corporation.

According to the second embodiment, as illustrated in FIG. 4, the soil analysis data on the targeted land is also obtained in addition to the profit estimation data and production planning data, and the production management data producing part provides production management data on the basis of these data, thereby enabling cultivation on new lands and cultivation of new crops on the basis of the plan supported by the data while estimating the profit.

### Third Embodiment

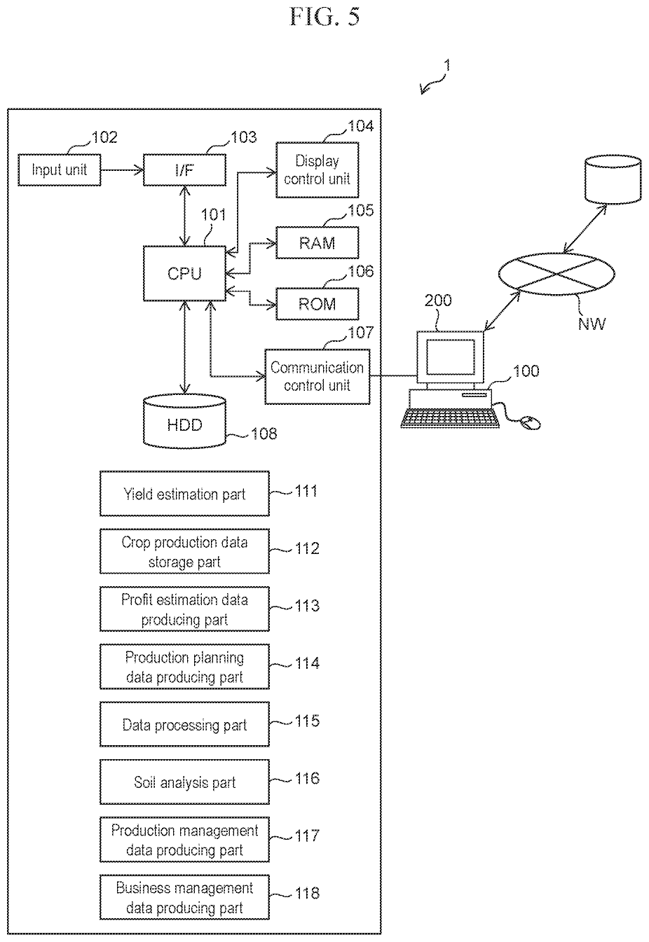

Next, the agriculture support system according to a third embodiment will be described with reference to FIG. 5, etc. FIG. 5 is a block diagram illustrating the overall configuration of the agriculture support system 1 according to the third embodiment. The system 1 of the third embodiment differs from those of the aforementioned embodiments in the computer program stored in the HDD 108 and the portion that is virtually implemented by the computer program. The constituent elements in FIG. 5 that are the same as those in FIG. 1 are denoted by the same reference numerals, and the overlapping descriptions will be omitted below.

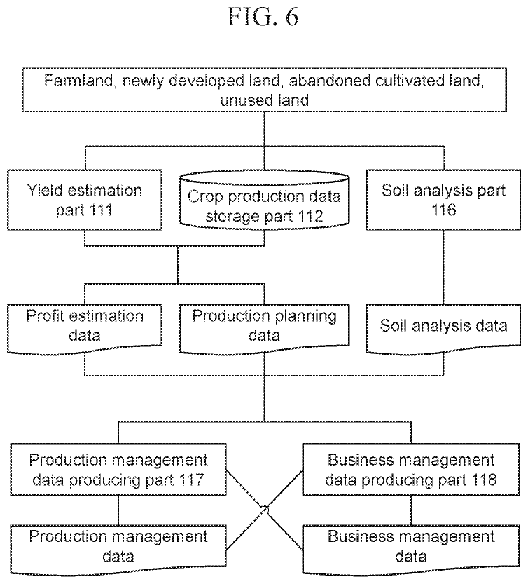

The computer program of the system 1 of the third embodiment includes a business management data producing unit 118 in addition to the same functions as those of the second embodiment. The business management data producing unit 118 has a function of producing business management data on farming business for implementing farming to produce a crop on a land in accordance with the selected crop and land, and the profit estimation data, production planning data, and soil analysis data on the crop to be produced on the land (see FIG. 6).

The business management data closely relates to the production management data. Therefore, the production management data producing part 117 produces production management data also referring to the business management data, while the business management data producing unit 118 produces business management data also referring to the production management data. Further, the production management data and business management data that are already produced may be processed to be output again by the data processing part 115 such that they are reflected on each other.

The present disclosure is not limited to the aforementioned embodiments, but may include various modifications. For example, the aforementioned embodiments are described in detail for easier understanding of the present disclosure, but the present disclosure is not necessarily limited to those including all the matters described. Further, the configuration of one embodiment may be partially replaced with that of another embodiment. In addition, the configuration of each embodiment may partially include or be replaced with another configuration, or be deleted.

### DESCRIPTION OF SYMBOLS

1 Agriculture support system
100 Computer
101 CPU
102 Input unit
103 Interface (I/F)
104 Display control unit
105 RAM
106 ROM
107 Communication control unit
108 Hard disk drive (HDD)
111 Yield estimation part
112 Crop production data storage part
113 Profit estimation data producing part
114 Production planning data producing part
115 Data processing part
116 Soil analysis part
117 Production management data producing part
118 Business management data producing part
200 Display
NW Network

What is claimed is:

1. An agriculture support system configured as a computer system, the agriculture support system comprising:
a data input unit;
a data storage unit; and
an arithmetic operation unit,
wherein the arithmetic operation unit includes:
a yield estimation part adapted to estimate a yield of a crop on the basis of data including characteristics of a land as an estimation target and environment of the land so as to output yield data;
a crop production data storage part adapted to store crop production data on crop production; and
a profit estimation part adapted to estimate profit for a case in which the crop is produced on the basis of the yield data and the crop production data so as to output profit estimation data.

2. The agriculture support system according to claim 1, wherein production planning is performed for the case in which the crop is produced on the basis of the yield data and the crop production data so as to output production planning data.

3. The agriculture support system according to claim 1, further comprising a soil analysis part adapted to analyze soil of the land so as to output soil analysis data.

4. The agriculture support system according to claim 3, further comprising a production management data producing part adapted to produce production management data for production management of the crop on the land on the basis of the profit estimation data and the soil analysis data.

5. The agriculture support system according to claim 4, further comprising a business management data producing part adapted to produce business management data for business management for a case in which the crop is produced on the land on the basis of the profit estimation data and the soil analysis data.

6. A method of estimating profit regarding agriculture, comprising:
estimating a yield of a crop on the basis of data including characteristics of a land as an estimation target and environment of the land so as to output yield data;
storing crop production data on crop production; and
estimating profit for a case in which the crop is produced on the basis of the yield data and the crop production data so as to output profit estimation data.

7. The method according to claim 6, further comprising
performing production planning for the case in which the crop is produced on the basis of the yield data and the crop production data, so as to output production planning data.

8. The method according to claim 6, further comprising
analyzing soil of the land so as to output soil analysis data.

9. The method according to claim 8, further comprising
producing production management data for production management of the crop on the land on the basis of the profit estimation data and the soil analysis data.

10. The method according to claim 8, further comprising
producing business management data for business management for a case in which the crop is produced on the land on the basis of the profit estimation data and the soil analysis data.

11. A computer-readable storage medium comprising instructions which, when executed by a computer, cause the computer to perform:
estimating a yield of a crop on the basis of data including characteristics of a land as an estimation target and environment of the land so as to output yield data;
storing crop production data on crop production; and
estimating profit for a case in which the crop is produced on the basis of the yield data and the crop production data so as to output profit estimation data.

12. The computer-readable storage medium according to claim 11, further comprising
performing production planning for the case in which the crop is produced on the basis of the yield data and the crop production data, so as to output production planning data.

13. The computer-readable storage medium according to claim 11, further comprising
analyzing soil of the land so as to output soil analysis data.

14. The computer-readable storage medium according to claim 13, further comprising
producing production management data for production management of the crop on the land on the basis of the profit estimation data and the soil analysis data.

15. The computer-readable storage medium according to claim 14, further comprising
producing business management data for business management for a case in which the crop is produced on the land on the basis of the profit estimation data and the soil analysis data.
